(19) United States
(12) Patent Application Publication
(10) Pub. No.: US 2024/0122880 A1
(43) Pub. Date: Apr. 18, 2024

(54) COMPOSITIONS FOR TREATING HYPERCHOLESTEROLEMIA AND PREPARATION METHODS THEREOF

(71) Applicants: UNIVERSITY OF SAN AGUSTIN, lloilo City (PH); DEL MONTE PHILIPPINES, INC., Taguig City (PH)

(72) Inventors: Jonel P. SALUDES, lloilo City (PH); Doralyn S. DALISAY, lloilo City (PH); Angelica Faith L. SUAREZ, lloilo City (PH); Angel Ann B. JUANILLO, lloilo City (PH); Dave Clark D. SISON, Cabuyao City (PH); Joey I. ORAJAY, Manolo Fortich (PH); Ma. Bella B. JAVIER, Taguig City (PH)

(73) Assignees: UNIVERSITY OF SAN AGUSTIN, lloilo City (PH); DEL MONTE PHILIPPINES, INC., Taguig City (PH)

(21) Appl. No.: 17/602,153

(22) PCT Filed: May 17, 2021
(86) PCT No.: PCT/PH2021/050013

§ 371 Date: Oct. 7, 2021

(30) Foreign Application Priority Data
Dec. 9, 2020 (PH) 12020050510

(51) Int. Cl. A61K 31/165; A61K 36/88

(52) U.S. Cl. CPC A61K 31/165; A61K 36/88; A61K 2236/15; A61K 2236/53

(57) ABSTRACT

The present invention relates to compositions for preventing or treating hypercholesterolemia, comprising an extract obtained from Ananas comosus, said extract comprising a therapeutically effective amount of N1, N10-Diferuloylspermidine, and preparation methods thereof. The invention provides for a natural product exhibiting HMG-CoA reductase and lipase enzyme inhibition that may be as effective as synthetic cholesterol-lowering drugs available in the market.

### TECHNICAL FIELD

The present invention relates to compositions for treating hypercholesterolemia comprising pineapple fruit extracts and preparation methods thereof.

### BACKGROUND OF THE INVENTION

Pineapples, Ananas comosus, are one of the most highly produced fruits in the Philippines. Despite its popularity in Filipino cuisine, there is a scarcity in scientific literature on the phytochemicals produced in the plant and the potential health benefits that could come from it. Should specific phytochemical compounds from pineapples be discovered to have medicinal properties, this would open financial doors for the agricultural industry.

Hypercholesterolemia is a condition wherein the cholesterol level in the blood is high. Excess cholesterol in the blood may form plaque in the walls of coronary arteries and lead to coronary heart disease. Patients with high cholesterol level are recommended to lower their intake of fatty food and may be prescribed synthetic drugs that lower cholesterol level. Such drugs used in treating hypercholesterolemia are hydroxymethylglutaryl-CoA (HMG-CoA) reductase inhibitors, also known as statins, and lipase inhibitors.

Commercially, pineapple products have been known for and marketed as having properties in aiding digestion and lowering cholesterol. For example, U.S. Pat. No. 6,509,372 discloses a method for inhibiting HMG-CoA reductase via a pharmaceutical composition of flavonoids that may be incorporated in pineapple juices. A study entitled “Hypolipidemic mechanisms of Ananas comosus L. leaves in mice: different from fibrates but similar to statins” by Xie et al. also discloses the lipid lowering properties of pineapple leaf extract that have mechanisms similar to statins.

The present invention demonstrates that pineapple extracts from pineapple flesh, core, and stem, comprising a specific compound N1, N10-Diferuloylspermidine, exhibit therapeutic properties that lower cholesterol. The invention provides for a natural product exhibiting HMG-CoA reductase and lipase enzyme inhibition that may be as effective as synthetic cholesterol-lowering drugs available in the market. This natural alternative to cholesterol-lowering drugs may lead to greater investments in cultivating pineapples and increases the value of the agricultural product.

### SUMMARY OF THE INVENTION

It is an object of the present invention to develop compositions for preventing or treating hypercholesterolemia comprising compounds exhibiting HMG-CoA reductase inhibition and lipase inhibition activities. It is another object of the present invention to provide natural therapeutic compositions from pineapple extract that would reduce the overall risk of cardiovascular diseases and prevent and treat such conditions. It is a further object of the present invention to provide methods for preparing compositions for treating hypercholesterolemia from Ananas comosus cores, flesh, or stems.

Accordingly, the present invention provides a composition for preventing or treating hypercholesterolemia, comprising an extract obtained from Ananas comosus, said extract comprising a therapeutically effective amount of N1, N10-Diferuloylspermidine.

Also provided are methods for preparing the composition set forth above from Ananas comosus cores, flesh, or stems.

In a related aspect, the composition comprising an extract obtained from Ananas comosus comprising a therapeutically effective amount of N1, N10-Diferuloylspermidine may be used in the manufacture of a medicament for preventing or treating hypercholesterolemia.

### BRIEF DESCRIPTION OF THE DRAWINGS

The accompanying drawings, which are included to provide a further understanding of the present invention, are incorporated herein to illustrate embodiments of the present invention. Along with the description, they also explain the principle of the present invention and are not intended to be limiting.

FIG. 1 shows the MALDI-TOF mass spectra of gel filtration chromatography (GFC) pineapple flesh (PAF) fractions 05-07 obtained using α-cyano-4-hydroxycinnamic acid (ACHCA) as the matrix.

FIG. 2 shows a High-Resolution Mass Spectrometry (HRMS) full mass spectrum of an active fraction obtained according to preferred methods of extraction and separation of the present invention.

FIG. 3 shows the tandem mass spectrometry (MS/MS) fragmentation of m/z 498.2599 [M+H]⁺ corresponding to N1, N10-Diferuloylspermidine, the active agent of the present invention.

FIG. 4 shows the molecular structure of N1, N10-Diferuloylspermidine, the hypocholesterolemic compound of the present invention.

### DETAILED DESCRIPTION OF THE INVENTION

The following are definitions of terms as used in the various embodiments of the present invention.

The term “pineapple flesh” or “Ananas comosus flesh” as used herein refers to as the soft edible tissue inside the fruit after removing the pineapple skin. This part is the commonly consumed part of the fruit.

The term “pineapple core” or “Ananas comosus core” as used herein refers to the hard part at the center of the inside the fruit that is surrounded by the pineapple flesh.

The term “pineapple stem” or “Ananas comosus stem” as used herein refers to the base of the pineapple plant that supports the plant leaves and fruit, and is connected to the roots.

The term “fraction” as used herein refers to purified or partially purified extracts.

The term “active fraction” as used herein refers to purified or partially purified extracts that comprises N1, N10-Diferuloylspermidine, a hypocholesterolemic compound that exhibits high HMG-CoA reductase and lipase inhibition properties.

The term “therapeutically effective amount” as used herein means the amount of N1, N10-Diferuloylspermidine in a composition, such as for example, an oral preparation, that when administered to an individual for treating a state, disease, disorder or condition associated with or caused by hypercholesterolemia is sufficient to effect such treatment. The therapeutically effective amount will vary depending on the particular state, disease, disorder or condition being treated and its severity and the age, weight, physical condition and responsiveness of the individual to be treated.

The present invention relates to compositions for preventing or treating hypercholesterolemia comprising an extract obtained from Ananas comosus, said extract comprising a therapeutically effective amount of N1, N10-Diferuloylspermidine, a hypocholesterolemic compound exhibiting HMG-CoA reductase and lipase inhibition. The compositions of the present invention produce at least 80% HMG-CoA reductase or lipase inhibition.

Preferably, the Ananas comosus extract in the present invention is obtained from at least one of pineapple flesh (PAF), pineapple cores (PAC), and pineapple stems (PAS).

Crude PAF, PAC, and PAS extracts comprise N1, N10-Diferuloylspermidine, the hypocholesterolemic compound exhibiting HMG-CoA reductase and lipase inhibition. Methods that may be used to obtain the crude pineapple extracts may include, but are not limited to, maceration, digestion, solvent extraction, reflux extraction, distillation, percolation, Soxhlet extraction, and pressurized liquid extraction.

In an embodiment of the present invention, the obtained crude PAF, PAC, and PAS extracts may be subsequently subjected to separation techniques to obtain active fractions of the crude extracts containing the hypocholesterolemic compound, N1, N10-Diferuloylspermidine. These separation techniques are performed to purify and concentrate the hypocholesterolemic compound in the crude extracts. In a preferred embodiment, the obtained pineapple flesh, core, and stem extracts are subjected to Gel Filtration Chromatography (GFC). Other separation techniques that may be employed may include, but are not limited to, high performance liquid chromatography, adsorption column chromatography, partition chromatography, membrane filtration, ion exchange chromatography, molecular distillation, gas chromatography, supercritical fluid chromatography, and molecular imprinted technology. Resulting fractions from these separation techniques are then subjected to lipase and HMG-CoA reductase inhibition assays to determine the fractions that contain the hypocholesterolemic compound for treating hypercholesterolemia. Fractions that demonstrate high inhibition of lipase and HMG-CoA reductase are considered as active fractions that may contain N1, N10-Diferuloylspermidine. Inhibition of HMG-CoA reductase and lipase activity lower cholesterol levels by slowing down cholesterol synthesis from the liver and reducing the hydrolysis of absorbable triglycerides. These properties are beneficial in treating hypercholesterolemia.

To demonstrate the presence of the hypocholesterolemic compound, N1, N10-Diferuloylspermidine, in pineapple fruits, mass spectra of pineapple extracts obtainable through preferred methods of extraction and separation are disclosed herein.

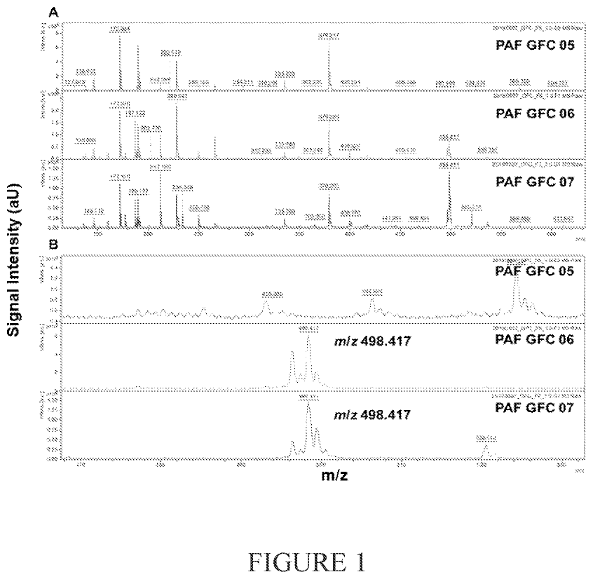

FIG. 1 MALDI-TOF mass spectra of obtained GFC PAF fractions 05-07 according to the disclosed extraction methods, using ACHCA as the matrix. FIG. 1A shows the full spectra from m/z 100600 and FIG. 1B shows a narrower mass range emphasizing the presence of signals at m/z 498 [M+H]⁺ in fraction GFC PAF 06.

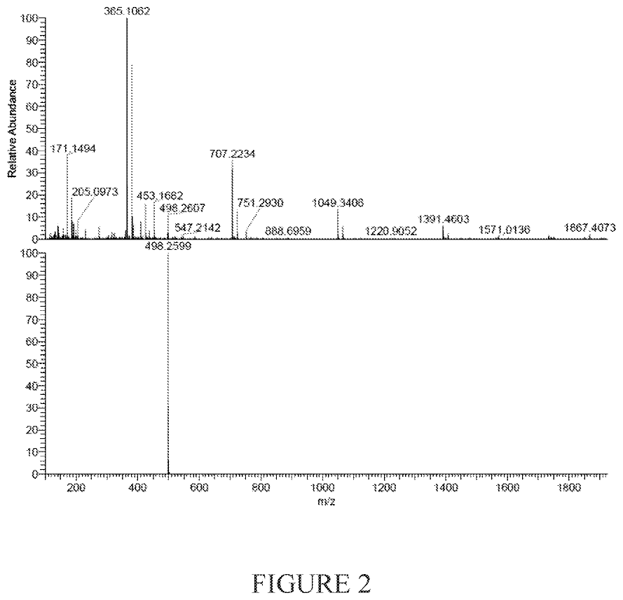

FIG. 2 shows an HRMS full mass spectrum of an active fraction, GFC PAF Fraction 07, obtained according to preferred methods of extraction and separation of the present invention. The major peak in the HRMS data is at m/z 498.2599 [M+H]⁺.

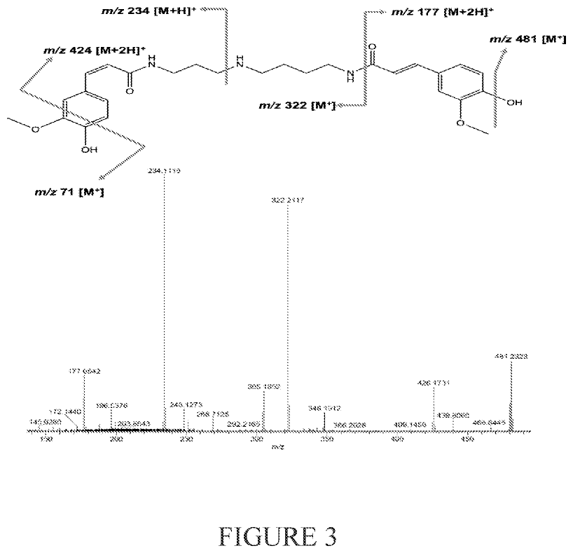

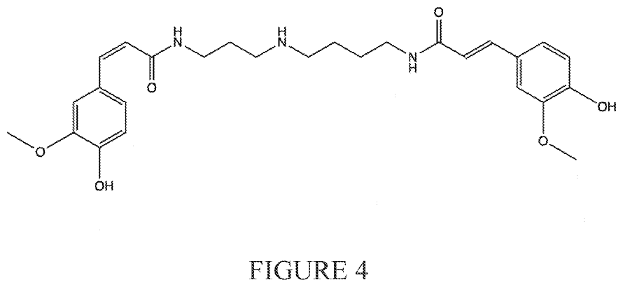

FIG. 3 shows the MS/MS fragmentation of m/z 498.2599 [M+H]⁺ corresponding to N1, N10-diferuloylspermidine, the hypocholesterolemic compound of the present invention. The fragmented ions at m/z 234, 322, 305, 176, and 481 in the positive mode shown by FIG. 3 corresponds to the compound N1, N10-diferuloyspermidine, the molecular structure of which is shown in FIG. 4. Based on this fragmentation data, mass spectra provided in FIG. 1 and FIG. 2 may indicate the presence of N1, N10-diferuloylspermidine in fractions GFC PAF 06 and GFC PAF 07.

FIG. 4 shows the molecular structure of N1, N10-Diferuloylspermidine (C₂₇H₃₅N₃O₆), the hypocholesterolemic compound in the compositions for treatment of hypercholesterolemia of the present invention. This compound N1, N10-diferuloylspermidine, with the measured mass of m/z 498.2599 [M+H]⁺, is determined to be the major compound present in GFC PAF Fraction 06, obtained according to preferred methods of extraction and separation of the present invention, which showed an 88.64±2.84% lipase inhibitory activity and a 100% HMG-CoA reductase inhibitory activity. This compound is a derivative of hydroxycinnamic acid amide, and a known compound reported to be distinct in the flesh region of the pineapple plant. The International Union of Pure and Applied Chemistry (IUPAC) name of this compound is (E)-3-(4-hydroxy-3methoxyphenyl)-N-[4-[3-[[(Z)-3-(4-hydroxy-3-methoxyphenyl)prop-2-enoyl]amino]propylamino]butyl]prop-2-enamide. Its IUPAC International Chemical Identifier (InChl) is 1S/C27H35N3O6/c1-35-24-18-20(6-1022(24)31)8-12-26(33)29-16-4-3-14-28-15-5-17-30-27(34)13-9-21-7-11-23(32)25(19-21)36-2/h613,18-19,28,31-32H,3-5,14-17H2,1-2H3,(H,29,33)(H,30,34)/b12-8+,13-9−. The compound's InChl key is IGHVUFYLAJSILE-UQXQTEIVSA-N. Its Canonical Simplified molecular-input line-entry system (SMILES) is COC1=C(C═CC(═C1)C═CC(═O)NCCCCNCCCNC(═O)C═CC2-CC(—C(C═C2)O)OC)O. Its Isomeric SMILES is COC1=C(C═CC(═C1)/C═C/C(═O)NCCCCNCCCNC(═O)/C═C\C2=CC(═C(C═C2)O)OC)O.

In an embodiment where the composition of the present invention uses crude PAF, PAC, and PAS extracts, the concentration of these crude extracts in the composition ranges from 5 to 100 mg/mL. In an embodiment where the composition of the present invention uses active fractions from the crude extracts, the concentration of these active fractions in the composition ranges from 1 to 10 mg/mL.

In a preferred embodiment, the composition of the present invention comprises a therapeutically effective amount of N1, N10-Diferuloylspermidine ranging from 0.1 to 30 percent by weight of the composition.

In preferred embodiments of the invention, the composition of the present invention may come in the form of a juice, a food additive, a food ingredient, a functional food, a medical food, a dietary supplement, a nutraceutical, a nutritional supplement or an oral preparation comprising the hypocholesterolemic compound, N1, N10-Diferuloylspermidine contained in pineapple extracts, used in treating hypercholesterolemia. In achieving these compositions of the present invention, pharmaceutically acceptable carriers or diluents may be added in formulation.

In a more preferred embodiment of the present invention, the composition in the form of a juice comprises a therapeutically effective amount of N1, N10-Diferuloylspermidine ranging from 0.1 to 100 mg/mL.

In accordance with another object of the present invention, methods for preparing compositions from Ananas comosus core, flesh, or stem extracts with a therapeutically effective amount of N1, N10-Diferuloylspermidine for treating hypercholesterolemia are also disclosed herein.

A method of preparing a composition for preventing or treating hypercholesterolemia using an Ananas comosus fruit extract comprises the steps of: obtaining at least one Ananas comosus fruit to extract from; separating Ananas comosus cores and Ananas comosus flesh of the at least one obtained Ananas comosus fruit; reducing the size of the separated Ananas comosus cores and Ananas comosus flesh; producing the Ananas comosus fruit extract by subjecting the size-reduced Ananas comosus cores and Ananas comosus flesh to a juice processor at about 4,000-8,000 rpm; subjecting the produced Ananas comosus fruit extract to an at least one filtration step; and concentrating N1, N10-Diferuloylspermidine in the filtered Ananas comosus fruit extract to a therapeutically effective concentration.

A method of preparing a composition for preventing or treating hypercholesterolemia using an Ananas comosus stem extract comprises the steps of: obtaining an at least one Ananas comosus stem to extract from; preparing the at least one Ananas comosus stem by removing stem skin and lignified portions; reducing the size of the at least one prepared Ananas comosus stem; obtaining the Ananas comosus stem extract by macerating the at least one size-reduced Ananas comosus stem in distilled water; decanting the obtained Ananas comosus stem extract from the at least one macerated Ananas comosus stem; subjecting the decanted Ananas comosus stem extract to at least one filtration step; and concentrating N1, N10-Diferuloylspermidine in the filtered Ananas comosus stem extract to a therapeutically effective concentration.

In a preferred embodiment, the methods of the present invention further comprise the step of formulating the composition by adding an at least one pharmaceutically acceptable carrier or diluent.

Preferred filtration steps for the disclosed preparation methods include, but are not limited to, muslin cloth filtration, gravity filtration, and vacuum filtration.

The step of concentrating N1, N10-Diferuloylspermidine in the filtered Ananas comosus extracts may be performed through liquid-liquid solvent extraction, Soxhlet extraction, rotary evaporation, supercritical fluid extraction, ultrasound-assisted, microwave-assisted extractions, and chromatographic techniques.

The extract obtained from Ananas comosus using the preparation methods disclosed, wherein the Ananas comosus extracts comprises a therapeutically effective amount of N1, N10-Diferuloylspermidine, may be used for the manufacture of a medicament for preventing or treating hypercholesterolemia.

Those skilled in the art will understand that they can freely combine all the features of the present invention disclosed herein. Other advantages and features of the present invention will be apparent from the accompanying drawings and the examples.

### EXAMPLES

### Example 1: Preparation of Plant Material

Twenty to twenty-five ripe pineapple fruits of green to yellow shell, as well as pineapple mature stems, were harvested. The samples were prepared and maintained at the temperature of 3-5° C. for a couple of days prior to analysis. The core was removed from the flesh using a pineapple slicer and the excess flesh from the skin was scraped using a kitchen knife. The flesh and core samples were cut into chunks and were put through a juice processor at approximately 4,000-8,000 rpm.

The stem skin and lignified portions were removed. The stem samples were minced into small pieces and macerated in three to seven L of distilled water. After around 11-20 hours of soaking, the saturated aqueous extract was decanted. The sample was then added with one to three L of distilled water and was allowed to soak again for another 11-20 hours.

The juice samples (core, flesh, or stem) were then subjected to filtration using five to layers of muslin cloth, and around one to five steps of cotton gravity filtration. Vacuum filtration was also performed in about three to five repeated steps using layers of well-stacked ordinary filter paper and cotton to further eliminate the fibers and clarify the juice. The clear juice samples (core, flesh, or stem) were distributed to 35-65 mL conical centrifuge tubes and were stored in −80° C. After overnight freezing, the samples were lyophilized. The dried pineapple crude extracts of core, flesh, or stem were dissolved in water to yield a concentration of 60 to 80 mg/mL.

### Example 2: Gel Filtration Chromatography (GFC) for Purification of Compounds

The compounds from the crude extract of pineapple core, flesh, or stem were separated according to its molecular size through GFC using 80 grams of Sephadex G-75 resin (pore size: 40-120 μm). The gel was conditioned in distilled H₂O for approximately 18 hours. The column measured bed height was between 30-55 cm with a radius of around 1.2-2.0 cm, which puts the bed volume at approximately 250-450 mL.

Three hundred fifty to six hundred fifty mg of pineapple flesh (PAF) extract in 0.7-1.3 mL of water at 0.9-1.5 mL/min flow rate yielded around 80-145 fractions of about 3.85-7.15 mL from 500-900 mL of water as the mobile phase. The void volume collected was between 50-90 mL which is about 20-40% of the bed volume.

The fraction was subjected to UV-Vis spectrophotometer for fraction pooling. The fractions were pooled based on their similarity in UV-Vis profile. The pooling was based on elution time wherein the total volume per major fraction was divided to its flow rate to obtain the time where the fraction is supposed to elute. Overall, a total of about 11.50 to 21.50 g of extract material for GFC was purified. All pooled fractions were numbered and tested for lipase and HMG-CoA reductase inhibitory activity.

### Example 3: Lipase Inhibition Assay

The lipase inhibition activities of the pineapple flesh, core, and stem crude extracts were determined by measuring the glycerol generated from the cleavage of triglycerides. The storage and preparation instruction were done according to the stated protocol of MAK, 046, Sigma Aldrich®, St. Louis, MO, USA.

Ten μL of the 100 mM Glycerol Standard with 990 μL of the Lipase Assay Buffer was diluted to prepare a 1 mM standard solution. Different volumes (0, 2, 4, 6, 8, 10, 12, 14 μL) of the standard stock solution were added to the wells in 96-well plates, respectively. Twenty μL of 750 mg/mL flesh, core, and stem at 100 mg/mL final concentration crude extracts were added to their respective wells.

Twenty μL of the GFC fractions of flesh, core, and stem were added to have 1 mg/mL to 10 mg/mL final concentration followed by the addition of 2 μL lipase positive control. Two μL of the lipase and Orlistat™ at 100 mg/mL and 10 mg/mL, respectively, were added to wells to serve as negative and positive control. All standards and samples were adjusted to 50 μL by adding Lipase Assay Buffer. A 100-μL reaction mix consisting of 93 μL Lipase Assay Buffer, 2 μL peroxidase substrate, 2 μL enzyme mix and 3 μL of lipase substrate was added to each well.

Absorbance was read at 570 nm every five minutes using CLARIOStar™ microplate reader at 37° C. incubation at slow kinetics mode for two hours. The percent lipase inhibition was calculated relative to the untreated lipase enzyme. One unit of lipase is the amount of enzyme that generated 1.0 μmol of glycerol from triglycerides per minute at 37° C.

Results of the lipase inhibition assays of pineapple extracts are summarized in Table 1 below.

TABLE 1
Lipase inhibition assay results of select
pineapple extracts and fractions

| | | Lipase inhibition |
|---|---|---|
| Sample | Concentration | activity |
| Untreated lipase (negative control) | — | 0% |
| Orlistat ™ (positive control) | 100 mg/mL | 100 ± 0.05% |
| Pineapple flesh (PAF) crude extract | 100 mg/mL | 100% |
| Pineapple core (PAC) crude extract | 100 mg/mL | 85.60 ± 1.92% |
| Pineapple stem (PAS) crude extract | 100 mg/mL | 70.06% |
| GFC PAF 03 | 10 mg/mL | 76.56 ± 4.40% |
| GFC PAF 06 | 10 mg/mL | 88.64 ± 2.84% |
| GFC PAC 01 | 10 mg/mL | 22.41 ± 1.57% |
| GFC PAC 02 | 10 mg/mL | 0% |
| GFC PAC 03 | 10 mg/mL | 30.64 ± 2.41% |
| GFC PAS 04 | 10 mg/mL | 64.69 ± 3.96% |
| GFC PAS 05 | 10 mg/mL | 57.27 ± 4.10% |

Lipase inhibition assay revealed that the pineapple flesh crude extracts exhibit hypolipidemic activity as demonstrated by lipase inhibition. GFC PAF Fraction 06 showed 88.64±2.84% inhibitory activity relative to the 100±0.05% inhibition of Orlistat™ at the same concentration, followed by GFC PAF Fraction 03 at 76.56±4.40%. These fractions, GFC PAF Fraction 06 and GFC PAF Fraction 03, exhibit the highest lipase inhibition activity among GFC fractions tested. Our findings revealed that the core section of the pineapple fruit has low lipase inhibitory activity compared to extracts from pineapple flesh and stem.

### Example 4: Hydroxyl-methylglutaryl-Coenzyme A (HMG-CoA) Reductase Inhibition Assay

The assay was based on the spectrophotometric measurement of the decrease in absorbance at 340 nm, which represents the oxidation of Nicotinamide Adenine Dinucleotide Phosphate (NADPH) by the catalytic subunit of 3-hydroxy-3-methylglutaryl CoA reductase in the presence of substrate HMG-CoA. All storage and reagent preparations were performed according to the protocol (CS1090, Sigma Aldrich®, St. Louis, MO, USA).

Assay buffer (1×) in the HMG-CoA assay kit was added to all samples. 184 μL for the blank, 182 μL for the activity control, 181 μL for the inhibition control and samples according to the manufacturer's instructions in the Sigma-Aldrich® CS1090 assay kit.

Pravastatin™ (2.1 μg/mL) positive control, 20 μL of g/mL flesh, core (100 mg/mL final concentration) and 20 μL of 50 mg/mL stem (5 mg/mL final concentration) crude extracts were added to its assay buffers. Lower reaction concentration of extracts (20 μL of 250 mg/mL to obtain 25 mg/mL final concentration of flesh and core crude extracts) was also tested against HMG-CoA reductase enzyme.

Four μL of NADPH and 12 μL HMG-CoA substrate were also added to the treatments. The HMG-CoA reductase enzyme (2 μL) was kept on ice until added to the activity, inhibition controls, and samples.

The absorbance at 340 nm was monitored for every 20 seconds for up to 10 minutes using CLARIOStar™ plate reader. One unit will convert 1 μmol of NADPH to NADP⁺ per one minute at 37° C.

Results of the HMG-CoA reductase inhibition assays of pineapple extracts are summarized in Table 2 below.

TABLE 2
HMG-COA reductase inhibition assay results
of select pineapple extracts and fractions

| | | | HMG-COA |
|---|---|---|---|
| | | | reductase |
| | | | inhibition |
| Sample | Concentration | | activity |
| Untreated HMGR (negative control) | — | | 0% |
| Pravstatin ™ (positive control) | 2.1 | μg/mL | 82% |
| Pineapple flesh (PAF) crude extract | 100 | mg/mL | 74.44 ± 1.73% |
| Pineapple core (PAC) crude extract | 100 | mg/mL | 70.50 ± 0.60% |
| Pineapple stem (PAS) crude extract | 5 | mg/mL | 100.00 ± 0.75% |
| GFC PAF 03 | 10 | mg/mL | 86.27% |
| GFC PAF 06 | 10 | mg/mL | 100% |
| GFC PAF 07 | 5 | mg/mL | 82.6% |
| GFC PAC 02 | 10 | mg/mL | 46.26 ± 8.79% |
| GFC PAC 04 | 10 | mg/mL | 100 ± 0.00% |
| GFC PAS 04 | 1.0 | mg/mL | 87.68% |
| GFC PAS 05 | 10 | mg/mL | 85.38% |
| GFC PAS 06 | 5.0 | mg/mL | 93.6% |

Among crude pineapple extracts, PAS crude extract exhibited the highest HMG-CoA reductase inhibition activity, demonstrating 100.00±0.75% inhibition. Among the GFC fractions, GFC PAF Fraction 06 and GFC PAC Fraction 04 exhibited the highest HMG-CoA reductase inhibition activity, demonstrating 100% and 100±0.00% inhibition, respectively.

### Example 5: MALDI-TOF Mass Spectrometry of Select Pineapple Flesh (PAF) GFC Fractions

GFC fractions that exhibited high inhibition of HMG-CoA reductase and lipase activity were subjected to MALDI-TOF mass spectrometry to examine the chemical profile of these fractions. GFC PAF 05, GFC PAF 06, and GFC PAF 07 were subjected to MALDI-TOF spectrometry using α-cyano-4-hydroxycinnamic acid (ACHCA) as the matrix. Chemical profiles obtained from these mass spectrometry results were studied to conduct further high-resolution mass spectrometry (HRMS) and determination of the chemical structure of potential active compounds in the fractions.

High resolution and high mass accuracy experiments were done using Linear Trap Quadrupole (LTQ) Orbitrap CL ETD mass spectrometer equipped with standard ESI ion source. Five μL of sample showing 100% HMG-CoA inhibition (GFC PAF Fraction 06) was flow injected at a rate of 50 μL/minute in 80% ACN/H2O 0.1% FA by Ultimate 3000 RSLC system from Dionex. The full-scan MS condition was m/z 100-2000 or 500-4000, resolution 60,000 at m/z 400. The target ions were sequentially isolated for MS/MS by LTQ. Electrospray voltage was maintained at 4 kV and capillary temperature was set at 275° C. HRMS and MS/MS data were analyzed to give the molecular structure of the compound.

1. A composition for preventing or treating hypercholesterolemia, comprising:
an extract obtained from Ananas comosus, said extract comprising a therapeutically effective amount of N1, N10-Diferuloylspermidine.

2. The composition of claim 1, wherein the extract is obtained from at least one of flesh, cores, and stems of Ananas comosus.

3. The composition of claim 1, wherein the therapeutically effective amount of N1, N10-Diferuloylspermidine ranges from 0.1 to 30 percent by weight of the composition.

4. The composition according to claim 1, wherein the composition is in the form of a juice, a food additive, a food ingredient, a functional food, a medical food, a dietary supplement, a nutraceutical, a nutritional supplement, or an oral preparation.

5. The composition of claim 1, wherein the therapeutically effective amount of N1, N10-Diferuloylspermidine ranges from 0.1 to 100 mg/mL in a juice.

6. The composition of claim 1, further comprising an at least one pharmaceutically acceptable carrier or diluent.

7. A method of preparing a composition for preventing or treating hypercholesterolemia using an Ananas comosus fruit extract, comprising the steps of:
a. obtaining an at least one Ananas comosus fruit to extract from;
b. separating Ananas comosus cores and Ananas comosus flesh of the at least one obtained Ananas comosus fruit;
c. reducing the size of the separated Ananas comosus cores and Ananas comosus flesh;
d. producing the Ananas comosus fruit extract by subjecting the size-reduced Ananas comosus cores and Ananas comosus flesh to a juice processor at about 4,000-8,000 rpm;
e. subjecting the produced Ananas comosus fruit extract to an at least one filtration step; and
f. concentrating N1, N10-Diferuloylspermidine in the filtered Ananas comosus fruit extract to a therapeutically effective concentration.

8. A method of preparing a composition for preventing or treating hypercholesterolemia using an Ananas comosus stem extract, comprising the steps of:
a. obtaining an at least one Ananas comosus stem to extract from;
b. preparing the at least one Ananas comosus stem by removing stem skin and lignified portions;
c. reducing the size of the at least one prepared Ananas comosus stem;
d. obtaining the Ananas comosus stem extract by macerating the at least one size-reduced Ananas comosus stem in distilled water;
e. decanting the obtained Ananas comosus stem extract from the at least one macerated Ananas comosus stem;
f. subjecting the decanted Ananas comosus stem extract to at least one filtration step; and
g. concentrating N1, N10-Diferuloylspermidine in the filtered Ananas comosus stem extract to a therapeutically effective concentration.

9. The method of claim 7, further comprising the step of adding an at least one pharmaceutically acceptable carrier or diluent.

10. (canceled)

11. The composition according to claim 2, wherein the composition is in the form of a juice, a food additive, a food ingredient, a functional food, a medical food, a dietary supplement, a nutraceutical, a nutritional supplement, or an oral preparation.

12. The composition according to claim 3, wherein the composition is in the form of a juice, a food additive, a food ingredient, a functional food, a medical food, a dietary supplement, a nutraceutical, a nutritional supplement, or an oral preparation.

13. The method of claim 8, further comprising the step of adding an at least one pharmaceutically acceptable carrier or diluent.
